(19) United States
(12) Patent Application Publication
(10) Pub. No.: US 2017/0105603 A1
(43) Pub. Date: Apr. 20, 2017

(54) ENDOSCOPE

(71) Applicant: OLYMPUS CORPORATION, Tokyo (JP)

(72) Inventor: Yoichi HOSAKA, Iruma-shi (JP)

(73) Assignee: OLYMPUS CORPORATION, Tokyo (JP)

(21) Appl. No.: 15/394,938

(22) Filed: Dec. 30, 2016

Related U.S. Application Data
(63) Continuation of application No. PCT/JP2015/052702, filed on Jan. 30, 2015.

(30) Foreign Application Priority Data
Jul. 2, 2014 (JP) 2014-137056

(51) Int. Cl. A61B 1/00; G02B 23/24; A61B 1/005

(52) U.S. Cl. CPC A61B 1/00039; A61B 1/00048; A61B 1/00052; A61B 1/00066; A61B 1/00032; A61B 1/005; G02B 23/2484

(57) ABSTRACT

An endoscope includes a flexible insertion section, a display section capable of displaying an endoscopic image, and a grasping section including a bottom portion with a ground contact surface, and to which the display section is fixed, where the insertion section extends downward from a rear surface of the grasping section with a proximal end of the insertion section connected to the grasping section, and the grasping section has a forward tilted posture with a top portion tilted to a front surface side opposite the rear surface.

### CROSS REFERENCE TO RELATED APPLICATION

This application is a continuation application of PCT/JP2015/052702 filed on Jan. 30, 2015 and claims benefit of Japanese Application No. 2014-137056 filed in Japan on Jul. 2, 2014, the entire contents of which are incorporated herein by this reference.

### BACKGROUND OF THE INVENTION

1. Field of the Invention

The present invention relates to an endoscope with a self-standing grasping section.

2. Description of the Related Art

Conventionally, endoscopes have been widely used in industrial and medical fields, for example. An examiner performs endoscopy by inserting an elongated insertion section into an examination target, and looking at an endoscopic image that is displayed on a display section. The examiner performs an insertion operation by operating an operation section with one hand, and holding the elongated insertion section with the other hand.

During endoscopy, an endoscope is sometimes used while being placed on a table or the like. Also, when the endoscope is not being used, the endoscope is sometimes temporarily placed on a table or the like. In these cases, the endoscope is required to have stability so as not to fall over easily. Particularly, because the endoscope includes an elongated insertion section, there is a problem that the endoscope easily falls over due to the weight of the insertion section.

Accordingly, Japanese Patent Application Laid-Open Publication No. 2012-242537 proposes an endoscope apparatus with a housing having both portability and stability. According to the proposed endoscope apparatus, the insertion section extends in a direction orthogonal to a shaft of a grip section. Also, to maintain stability of the apparatus, the housing of the endoscope apparatus includes the grip section that is formed to tilt from a front side to a rear surface side, and a base section having a ground contact surface, and when viewed from the side, the housing is substantially triangular.

### SUMMARY OF THE INVENTION

According to an aspect of the present invention, there may be provided an endoscope including a flexible insertion section, a display section capable of displaying an endoscopic image, and a grasping section including a bottom portion with a ground contact surface, and to which the display section is fixed, where the insertion section extends downward from a rear surface of the grasping section with a proximal end of the insertion section connected to the grasping section, and the grasping section has a forward tilted posture with a top portion tilted to a front surface side opposite the rear surface.

### BRIEF DESCRIPTION OF THE DRAWINGS

FIG. 1 is a front view of an endoscope according to an embodiment of the present invention;

FIG. 2 is a side view of the endoscope according to the embodiment of the present invention;

FIG. 3 is a perspective view of the endoscope according to the embodiment of the present invention, seen from obliquely behind;

FIG. 4 is a perspective view of the endoscope according to the embodiment of the present invention, showing a state where a cover for battery replacement is partially opened; and

FIG. 5 is a bottom view of the endoscope according to the present embodiment.

### DETAILED DESCRIPTION OF THE PREFERRED EMBODIMENT(S)

Hereinafter, an embodiment of the present invention will be described with reference to the drawings.

### (Overall Structure)

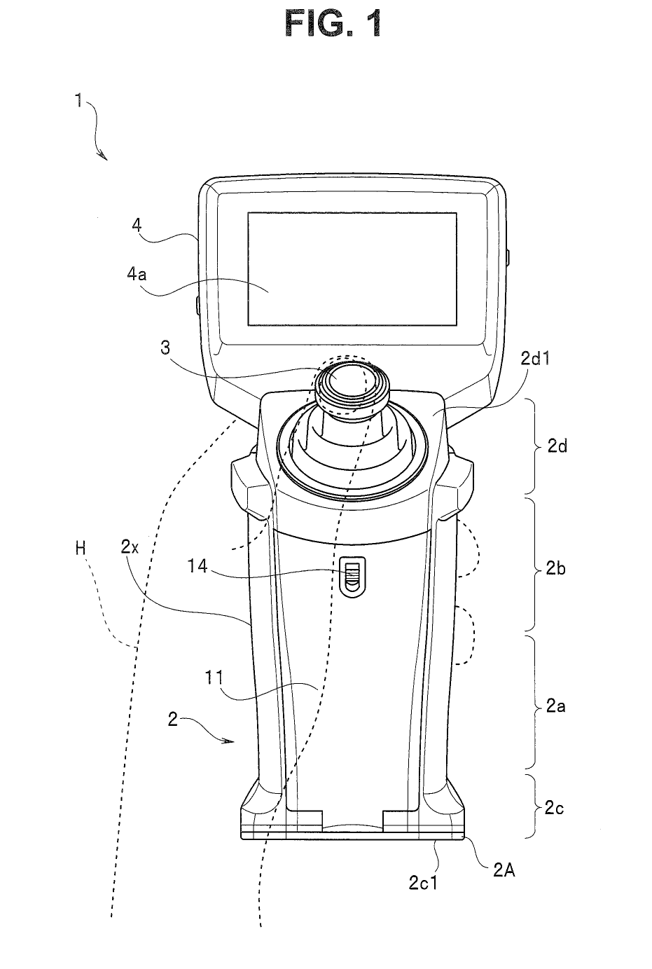

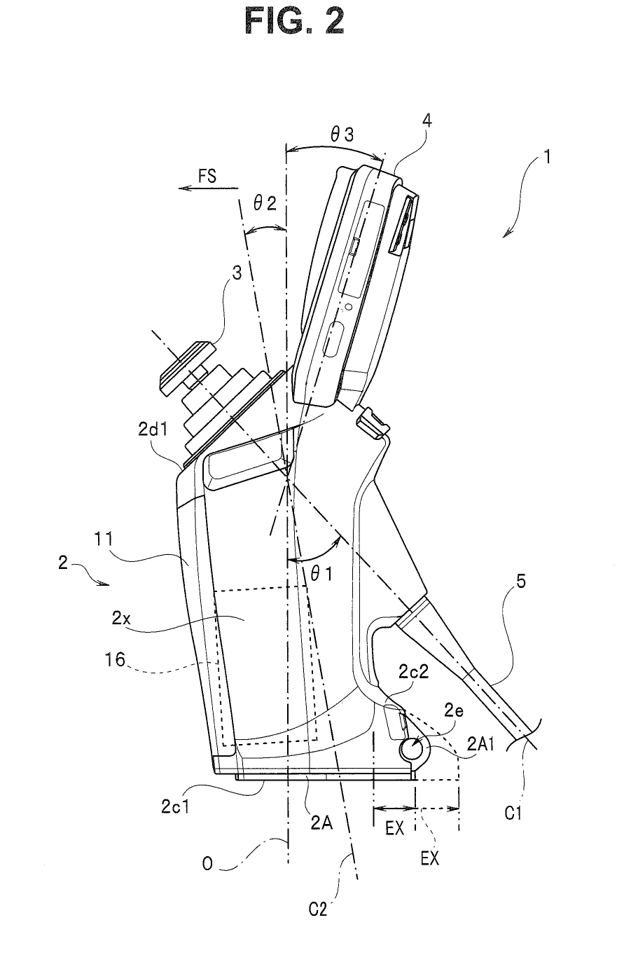

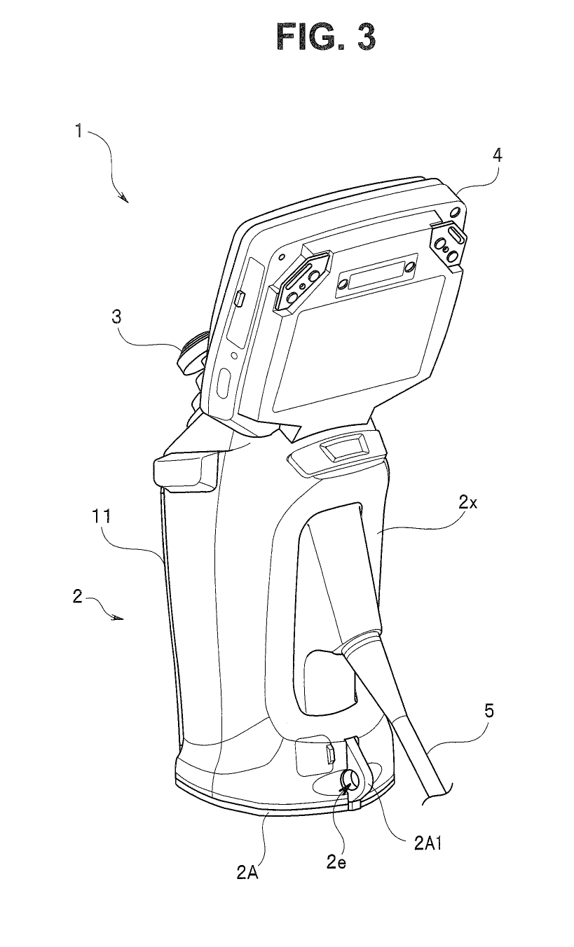

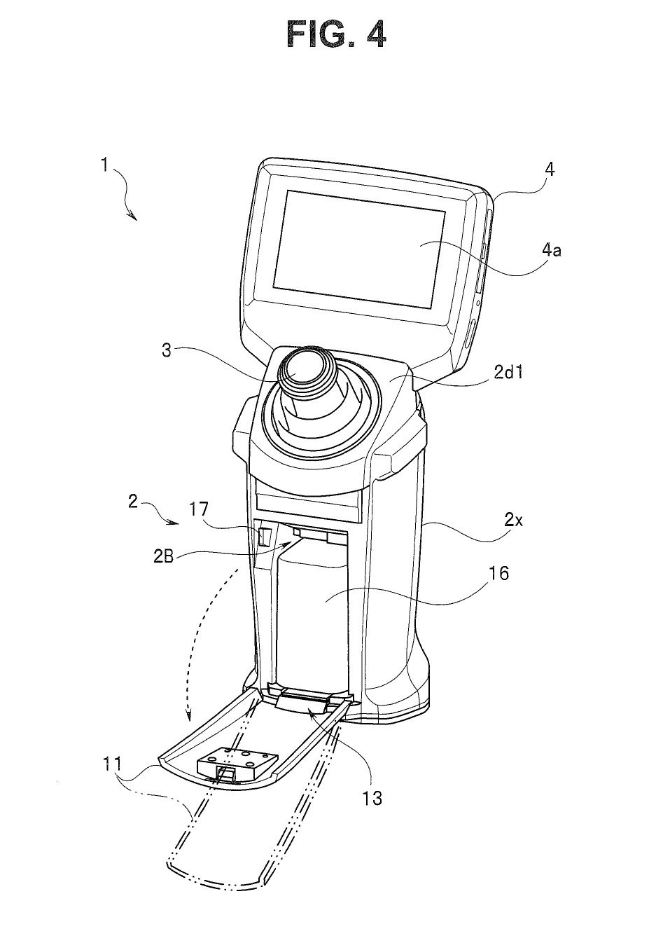

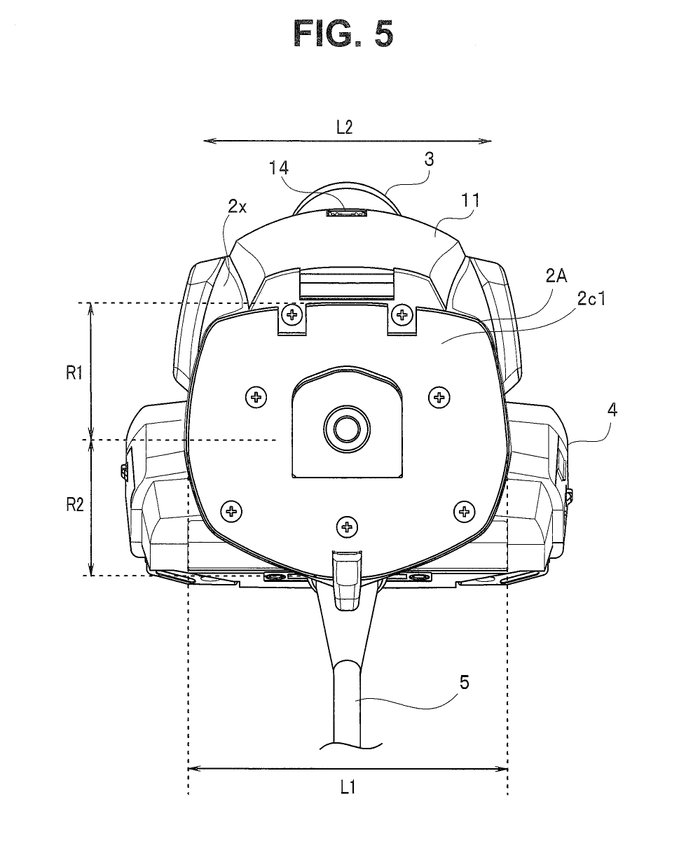

FIG. 1 is a front view of an endoscope according to a present embodiment. FIG. 2 is a side view of the endoscope according to the present embodiment. FIG. 3 is a perspective view of the endoscope according to the present embodiment, seen from obliquely behind. FIG. 4 is a perspective view of the endoscope according to the present embodiment, showing a state where a cover for battery replacement is partially opened. FIG. 5 is a bottom view of the endoscope according to the present embodiment.

An endoscope 1 is a battery-driven endoscope including a grasping section 2, a joystick 3, a display section 4 that is capable of displaying an endoscopic image, and an insertion section 5.

The grasping section 2 has a substantially cylindrical trunk section. The diameter of a slightly lower side portion 2a than a center portion of the grasping section 2 is slightly reduced than the diameter of a slightly upper side portion 2b than the center portion of the grasping section 2 so that when a user, who is an examiner, grasps the grasping section 2 with one hand H (right hand or left hand) as shown by dotted lines in FIG. 1, the user is able to firmly grasp the grasping section 2. The lower side portion 2a and the upper side portion 2b form the trunk section.

A housing 2x of the grasping section 2 includes an extending portion at the upper side portion 2b of the grasping section 2, and the display section 4 is fixedly provided on the extending portion. That is, the housing 2x is a housing for the grasping section 2 and the display section 4.

Additionally, in the following, a side facing a display surface 4a of the display section 4 when a user is grasping the grasping section 2 with one hand and looking at an endoscopic image, which is an image being examined, displayed on the display section 4 will be referred to as a front side of the grasping section 2. In FIG. 2, an arrow FS indicates the front side of the display section 4.

Furthermore, the grasping section 2 includes a bottom portion 2c that can be placed on a table such as a desk. The bottom portion 2c has an increased diameter and includes a flat bottom surface 2c1 so that the grasping section 2 does not easily fall over when placed on a table. The bottom surface 2c1 is a ground contact surface to a table or the like. That is, the grasping section 2 of the endoscope 1 is capable of self-standing on a table or the like. The grasping section 2 is to self-stand on a table or the like with the longitudinal direction of the grasping section 2 substantially coinciding with the vertical direction.

Additionally, in this case, the bottom surface 2c1 is a bottom surface of a bottom plate member 2A, as a slip prevention member, which is fixed to the housing 2x of the grasping section 2 by a screw or the like. The housing 2x of the grasping section 2 is made of resin. The bottom plate member 2A is made of a member with high friction coefficient, and is a rubber member in this case.

A top portion 2d of the grasping section 2 includes an inclined surface 2d1, which is a flat surface that is inclined toward the front side. The joystick 3, which is an operator, is provided protruding in a direction orthogonal to the inclined surface 2d1. That is, the joystick 3 as an operator is provided on the top portion 2d side of the grasping section 2. Moreover, when seen from the front surface side, the width of the grasping section 2 is greater on the top portion 2d side than on the bottom portion 2c side.

The size of the grasping section 2 is such that a user is able to grasp the grasping section 2 with one hand, and to operate the joystick 3 with a thumb while grasping the grasping section 2. As shown by the dotted lines in FIG. 1, the user is able to operate the joystick 3 with a thumb while grasping the grasping section 2 with a hand H.

The display section 4 is disposed while being tilted obliquely rearward from the top portion 2d of the grasping section 2 so that the display surface 4a faces the front side of the grasping section 2 while being slightly upward. That is, the display section 4 is tilted toward the rear surface side of the grasping section 2.

As shown in FIGS. 2 and 3, the flexible elongated insertion section 5 is provided on a rear surface of the grasping section 2, extending obliquely downward from the rear surface. That is, the grasping section 2 has a shape that allows a user to grasp the grasping section 2 with one hand, has the display section 4 fixed to the top portion 2d of the grasping section 2, and has the insertion section 5 extending from the rear surface with a proximal end of the insertion section 5 connected to the grasping section 2.

Furthermore, a retaining hole 2e for retaining the insertion section 5 by engaging with a distal end portion of the insertion section 5 is provided in a lower part on the rear surface side of the bottom portion 2c of the grasping section 2. The retaining hole 2e is formed to an upward extending portion 2A1, which is a part of the bottom plate member 2A and which protrudes upward. A user may cause the distal end portion of the insertion section 5 to be engaged with and retained in the retaining hole 2e by passing the distal end portion of the insertion section 5 through the retaining hole 2e. The retaining hole 2e is formed as a member with a great frictional force, and retains the distal end portion of the insertion section 5 in such a way that the distal end portion does not fall out of the hole.

A cover 11 is provided on a front surface of the grasping section 2, and is a cover for covering a battery accommodating section 2B that is disposed on the inside of the front surface side of the grasping section 2. That is, the battery accommodating section 2B capable of accommodating a battery 16 is provided in the grasping section 2, below the joystick 3.

Moreover, a hinge 13 for opening and closing the cover 11 is provided in the bottom portion 2c of the grasping section 2. As shown in FIG. 4, the cover 11 is enabled by the hinge 13 to be opened by having a top portion tilted forward on the front surface side of the grasping section 2. In other words, the cover 11 may open from the upper side in the longitudinal direction of the grasping section 2 toward the lower side.

That is, the cover 11 is a cover for covering the battery accommodating section 2B, and may be opened by having the top portion of the cover 11 tilted toward the front side while having a lower part of the cover 11 pivotally supported by the hinge 13 at a lower part of the grasping section 2.

A lock release operation member 14 for releasing a locked state in which the cover 11 is closed is provided on the top portion of the cover 11.

Furthermore, as shown in FIG. 5, the bottom surface of the bottom plate member 2A is the bottom surface 2c1 of the bottom portion 2c of the grasping section 2. The bottom surface 2c1 serves as a ground contact surface when the grasping section 2 of the endoscope 1 is placed on a desk, ground, a table or the like.

As described above, the grasping section 2 is a grasping section including the bottom portion 2c having a flat ground contact surface, and to which the display section 4 is fixed, and the insertion section 5, the proximal end of which is connected to the grasping section, extends downward from the rear surface of the grasping section 2, and the grasping section 2 has a forward tilted posture with the top portion tilted to the front surface side opposite the rear surface.

### (Structure for Allowing Stable Self-Standing of Grasping Section)

Next, description will be given on a structure for allowing stable self-standing of the grasping section 2 of the endoscope 1 including the elongated insertion section 5 when the grasping section 2 is placed on a table or the like.

### 1) Forward Tilted Posture of Grasping Section 2

First, the grasping section 2 of the endoscope 1 has a forward tilted posture so as to be able to self-stand. That is, when the grasping section 2 is placed on the ground, a table or the like, the grasping section 2 is inclined forward. An axis C2 of the upper side portion 2b and the lower side portion 2a forming the trunk section of the grasping section 2 is tilted toward the front surface side with respect to an axis 0 in the vertical direction.

As shown in FIG. 2, when viewed from the side, the axis C2 of the trunk section is inclined toward the front side at an angle θ2 with respect to the axis O in the vertical direction that is orthogonal to the bottom surface 2c1. The angle θ2 is smaller than an angle θ1 described later. The angle θ2 is an angle within a range of 5 degrees to 30 degrees, and is preferably about 10 degrees.

The grasping section 2 placed on a table or the like is sometimes pulled toward the rear surface side by the weight of the insertion section 5. The grasping section 2 has the forward tilted posture so that the top portion 2d protrudes toward the front surface side opposite the rear surface side in the pull direction. The forward tilted posture of the grasping section 2 causes, with respect to a force of pulling the grasping section 2 downward toward the rear surface side by the weight of the insertion section 5, a moment toward the front surface side opposite the rear surface side to act on the grasping section 2.

As described above, because the grasping section 2 has a forward tilted posture by which the top portion 2d is tilted toward the front surface side opposite the rear surface side, the grasping section 2 does not easily fall over when placed on the ground, a table or the like.

2) Insertion Section 5 Extending Downward from Rear Surface Side of Grasping Section 2

As shown in FIG. 2, the insertion section 5 extends, from the rear surface opposite the front surface of the grasping section 2, downward with a predetermined angle θ1 to the axis O in the vertical direction instead of a direction that is orthogonal to the axis O of the grasping section 2. The predetermined angle θ1 is an angle within a range of 20 degrees to 50 degrees, and preferably, the angle is within a range of 40 degrees to 45 degrees.

As shown in FIG. 2, an axis C1 of the insertion section 5 extending from the rear surface side of the grasping section 2 coincides with the axis of the joystick 3. In other words, the axis of the shaft portion of the joystick 3 and the axis in the extending direction of the proximal end portion of the insertion section 5 are coaxial.

According to the structure described above, the insertion section 5 is positioned at a lower part of the grasping section 2, and the center of gravity of the grasping section 2 is low, and thus, the grasping section 2 is stable and does not easily fall over.

Moreover, because the trunk section of the grasping section 2 has a forward tilted posture and is able to self-stand, and also, the insertion section 5 extends from the rear surface side of the grasping section 2, the area of the bottom surface of the grasping section 2 may be reduced, and the entire grasping section 2 may be miniaturized.

### 3) Extending Portion 2c2 Extending From Rear Surface Side of Bottom Portion 2c of Grasping Section 2

Furthermore, as shown in FIG. 2, the bottom portion 2c of the grasping section 2 includes an extending portion 2c2 extending by an amount of extension EX from the rear surface side of the grasping section 2. Accordingly, when viewing the endoscope 1 from the side, the grasping section 2 forms an L shape with the trunk section including the lower side portion 2a and the upper side portion 2b and the extending portion 2c2 of the bottom portion 2c.

The grasping section 2 is pulled downward from the rear surface side by the weight of the insertion section 5, but is prevented by the extending portion 2c2 of the bottom portion 2c from easily falling over even when pulled downward from the rear surface side.

Additionally, as shown by a dotted line, the amount of extension EX of the extending portion 2c2 may be increased.

### 4) Shape of Bottom Surface 2c1 of Bottom Portion 2c

As shown in FIG. 5, the bottom surface 2c1, which is the ground contact surface, has a left-right symmetric shape with respect to an axis in the front-back direction from the front surface side to the rear surface side. Also, the bottom surface 2c1, which is the ground contact surface, is shaped such that a ground contact area R1 on the front surface side and a ground contact area R2 on the rear surface side are different from each other. The ground contact area R2 on the rear surface side of the bottom surface 2c1 is formed to be greater than the ground contact area R1 on the front surface side of the bottom surface 2c1.

As a result, when the grasping section 2 is pulled downward from the rear surface side by the weight of the insertion section 5, a great friction may be caused by a contact region of the ground contact area R2 on the rear surface side of the bottom surface 2c1.

Furthermore, as shown in FIG. 5, a width L1 on the rear surface side of the bottom surface 2c1 is formed to be greater than a width L2 on the front surface side. That is, the bottom surface 2c1 of the bottom portion 2c is wider on the rear surface side than on the front surface side. Accordingly, the grasping section 2 is stable and does not easily fall over when the grasping section 2 is pulled downward from the rear surface side by the weight of the insertion section 5.

Therefore, the grasping section 2 does not easily fall over even when pulled downward at the angle θ1 by the weight of the insertion section 5 and pulled slightly in the left-right direction when the endoscope 1 is placed on a table or the like.

5) Bottom Plate Member 2A which is Rubber Member

The bottom plate member 2A, as a slip prevention member, which is a rubber member with high friction coefficient is provided on the bottom portion 2c. The bottom plate member 2A, which is a rubber member, is heavier than the trunk section of the grasping section 2, which is formed by a resin housing. That is, the bottom portion 2c is provided with the bottom plate member 2A of a material that is heavier than that of the housing of the grasping section 2. Accordingly, the center of gravity of the grasping section 2 is made low, and the grasping section 2 is prevented from easily falling over.

Furthermore, because the bottom surface 2c1 is a rubber member, the friction coefficient is high. Accordingly, when the grasping section 2 is placed on a table or the like, the bottom surface of the bottom plate member 2A does not easily slip, and the grasping section 2 is able to firmly self-stand without moving on the table or the like.

The grasping section 2 is pulled from the rear surface side by the weight of the insertion section 5, but because the bottom surface 2c1 does not easily slip due to the friction of the rubber, the grasping section 2 is less likely to fall off a table or the like.

Additionally, the material of the bottom plate member 2A as a slip prevention member may be elastomer or foam material instead of rubber. Furthermore, if the grasping section 2 of the endoscope 1 is to be placed on an iron plate, a member with a magnet may also be used.

### 6) Position of Battery 16

The battery 16 has a cuboid shape, and the battery 16 is accommodated inside the battery accommodating section 2B with the longitudinal axis of the battery 16 substantially in parallel with the axis in the vertical direction. Also, as shown by a dotted line in FIG. 2, the battery 16 accommodated inside the battery accommodating section 2B is positioned on the bottom portion 2c side of the grasping section 2, near the front surface side.

Because the battery 16 is on the front surface side of the grasping section 2, a moment toward the front surface side opposite the rear surface side is caused to act on the grasping section 2, with respect to a force of pulling the grasping section 2 downward toward the rear surface side by the weight of the insertion section 5.

The battery 16 is relatively heavy, and because the battery 16 is positioned on the bottom portion 2c side of the grasping section 2, the center of gravity of the grasping section 2 is low. Accordingly, the stability of the grasping section 2 at the time of the grasping section 2 being placed on a table or the like may be increased.

Furthermore, the battery 16 is accommodated inside the battery accommodating section 2B in a so-called upright manner, and thus, the area of the bottom surface 2c1 may be made small. ps 7) Display Section 4 Inclined toward Rear Surface Side of Grasping Section 2

As shown in FIG. 2, the display section 4 is fixed while being inclined toward the rear surface side at a predetermined angle θ3 with respect to the axis 0 of the grasping section 2. As a result, a weight balance is maintained with respect to the forward tilted posture of the grasping section 2, and high stability is achieved on a table or the like.

Additionally, because the display section 4 is inclined toward the rear surface side with respect to the axis 0 of the grasping section 2, an operating person may easily view the screen of the display section 4.

As described above, according to the endoscope of the embodiment described above, because the grasping section may be placed on a table or the like in a self-standing manner, a wide space is not required for placement of the endoscope, and also, the endoscope does not easily fall over, and thus, the endoscope may be used in various places.

An examiner may perform endoscopy with the grasping section 2 placed on a table or the like, or may temporarily place the grasping section 2 on a table or the like during examination, and at the time of storing the endoscope 1, a small space suffices for placement.

Furthermore, according to the embodiment described above, the area of the bottom surface 2c1 of the grasping section 2 may be reduced, and the endoscope may be miniaturized.

According to the present invention, an endoscope which does not easily fall over and the space for placement of which may be relatively small may be provided.

The present invention is not limited to the embodiment described above, and various modifications, alterations and the like are possible within the scope of the present invention.

What is claimed is:

1. An endoscope comprising:
a flexible insertion section;
a display section capable of displaying an endoscopic image; and
a grasping section including a bottom portion with a ground contact surface, and to which the display section is fixed, where the insertion section extends downward from a rear surface of the grasping section with a proximal end of the insertion section connected to the grasping section, and the grasping section has a forward tilted posture with a top portion tilted to a front surface side opposite the rear surface.

2. The endoscope according to claim 1, wherein the bottom portion includes an extending portion extending on the rear surface side.

3. The endoscope according to claim 2, wherein an area of the ground contact surface on the rear surface side is greater than an area of the ground contact surface on the front surface side.

4. The endoscope according to claim 2, wherein a width of the ground contact surface on the rear surface side is greater than a width of the ground contact surface on the front surface side.

5. The endoscope according to claim 3, comprising a slip prevention member provided on the bottom portion of the grasping section,
wherein the slip prevention member includes the ground contact surface.

6. The endoscope according to claim 5, wherein the slip prevention member is a rubber member.

7. The endoscope according to claim 1, comprising a joystick as an operator provided on an upper portion side of the grasping section,
wherein an axis of a shaft portion of the joystick and an axis in an extending direction of the proximal end of the insertion section are coaxial.

8. The endoscope according to claim 1,
wherein the display section is fixed at the top portion of the grasping section, and
the display section is inclined toward the rear surface side.

9. The endoscope according to claim 1, comprising a battery accommodating section that is provided in the grasping section and that is capable of accommodating a battery,
wherein the battery is positioned near the front surface side of the grasping section when accommodated inside the battery accommodating section.

10. The endoscope according to claim 2, comprising a retaining hole configured to retain the insertion section, on the rear surface side of the bottom portion of the grasping section.
